(19) United States
(12) Patent Application Publication
(10) Pub. No.: US 2011/0117577 A1
(43) Pub. Date: May 19, 2011

(54) MICROFLUIDIC SYSTEM FOR TRAPPING AND DETECTION OF A BIOLOGICAL ENTITY IN A SAMPLE

(75) Inventors: Julien Reboud, (US); Hongmiao Ji, Singapore (SG); Kokchuan Lee, Singapore (SG); Huimin Lim, Singapore (SG)

(73) Assignee: Agency for Science, Technology and Research

(21) Appl. No.: 12/908,553

(22) Filed: Oct. 20, 2010

(30) Foreign Application Priority Data
Oct. 20, 2009 (SG) 200906998-0

(51) Int. Cl. G01N 33/574; C12M 1/34; H05K 13/00

(52) U.S. Cl. 435/7.23; 435/287.1; 216/13; 29/825

(57) ABSTRACT

According to embodiments of the present invention, a microfluidic system for detecting a biological entity in a sample volume is provided. The microfluidic system includes: an inlet configured to receive the sample volume; at least one microchannel in fluid communication with the inlet; a magnetic trapping region comprising the at least one microchannel; at least one detection region in fluid communication with the magnetic trapping region for detecting the biological entity to be detected; and at least one outlet in fluid communication with the at least one detection region.

### CROSS-REFERENCE TO RELATED APPLICATIONS

This application claims the benefit of priority of Singapore application No. 200906998-0, filed 20 Oct. 2009, the content of it being hereby incorporated by reference in its entirety for all purposes.

### TECHNICAL FIELD

Various embodiments relate to a microfluidic system for detecting a biological entity in a sample volume and a method of forming the microfluidic system. Various embodiments further relate to an assembly including the microfluidic system for detecting a biological entity and a method for detecting a biological entity using the microfluidic system.

### BACKGROUND

Cancer is a leading cause of death worldwide, with more than 7.6 million deaths in 2007 (American Cancer Association, 2008). It represents a tremendous burden on patients, families and societies, with long and painful therapies and remissions. Furthermore, clinicians lack the precise tools to assist them in tailoring the dosage of treatments, usually laden with potentially serious side effects (radiation, chemotherapies), for the patients. For example, conventional imaging techniques and biopsies can only detect tumors that have reached a certain size; hence it may be difficult to establish or ascertain the complete remission of the disease. Moreover, these techniques can be painfully invasive and/or costly.

In 2008, the United States Food and Drug Administration (FDA) cleared the way for a system (CellSearch™, Veridex) which reports the level of circulating tumour cells (CTCs) in metastatic breast cancer patients. CTCs are tumor cells that have detached from the primary tumor site and are circulating in the bloodstream. The number of CTCs in the blood is a clear indicator of the aggressiveness of the cancer as well as the efficacy of the therapy being applied (Pantel, K. et al., “Detection, clinical relevance and specific biological properties of disseminating tumour cells”, Nat Rev Cancer. 2008, 8(5):329-40). Hence, CTCs represent a significant biomarker in early diagnosis and therapy monitoring with a potential to allow clinicians to cater therapies to specific patients and diseases as their number can be assessed in a relatively non-invasive manner, for example by blood drawing.

The detection of CTCs is generally based on the presence of the specific epithelial marker, epithelial cell adhesion molecule (EpCAM), on their surface. The technical challenge associated with the detection of CTCs lies in the fact that only a few such cells can be found in milliliters of blood, amongst millions of white blood cells and billions of red blood cells, even for patients at an advanced stage of cancer, who would be expected to have an increased number of CTCs in their bloodstream.

Conventionally, purification to isolate the CTCs is performed via complex magnetic separation steps in tubes, using beads coated with an antibody specific to the EpCAM receptor. The nature of the cells is then confirmed via fluorescent staining of cancer markers (cytokeratins for example) and lymphocytes receptors (CD45 for example) (Cristofanilli M. et al., “Circulating Tumor Cells, Disease Progression, and Survival in Metastatic Breast Cancer”, N Engl J Med. 2004, 351:781-91). The CellSearch™ system has automated this procedure into a reproducible assay. However, the procedure requires a sample transfer between two separate machines, which may cause contamination or loss of cells during the transfer. Besides, the sample even after magnetic purification, still contains a lot of other cells, necessitating a labor-intensive manual inspection of the stained cells by a trained specialist to establish the nature of the CTCs. Hence, the procedures are complex and costly, which prevent them from being used as frequent tests for therapy monitoring, although they are used for early diagnosis and prognosis.

Conventional detection systems for CTCs are generally bulky and expensive equipments, which are used to perform labor-intensive procedures. Although miniaturized microfluidic platforms are available, these still rely on detailed optical characterization via fluorescent markers. This hinders their integration into complete walk-away or portable systems, which would otherwise decrease costs and hassle and streamline medical access for cancer patients, for example gastric cancer patients.

Label-free detection techniques, such as surface plasmon resonance (SPR), quartz crystal microbalance (QCM) or impedance spectroscopy, provide automation and integration to rare cell detection. These techniques can be used with either flow-through devices, similar to Fluorescence Activated Cell Sorting (FACS), or batch-based devices. Flow-through systems (Wang Y-N. et al, “On-chip counting the number and the percentage of CD4+ T lymphocytes”, Lab Chip 2008, 8, 309-315; Roeser T. et al., “Lab-on-chip for the Isolation and Characterization of Circulating Tumor Cells”, Proceedings of the 29th Annual International Conference of the IEEE EMBS, 2007, 6446-6448) are capable of individual cell detection which enables counting of cells in the sample, but these systems are laden with long processing times and also require a high purity of cells at the detection module, thereby transferring the burden to the sample preparation module to prepare high purity samples. In addition, flow-through fluidics are inefficient for large volume processing due to the long processing times and low sensitivity.

Batch-based systems enable the incorporation of a specific selection of cells inside a detection module and batch processing of samples, thus reducing the process time. However, these systems are not capable of counting cells, but merely provide semi-quantitative levels. Nevertheless, for therapy monitoring, a resolution of 1 cell in the number obtained is not required.

While the batch-based methods can process bigger volumes of samples, these are still orders of magnitude below the samples used for CTC detection. Generally, a chamber containing a microelectrode array (MEA) of 1-10 electrodes for detecting<10 CTCs, can accommodate only about 1-5 μl of sample. In addition, samples are generally transferred through pipetting or tubes, which may lead to significant loss of cells, for example unspecific adhesion of cells to the walls of the transferring apparatus or cells trapped at the connections or interfaces, which may be as high as 70% loss.

Label-free systems to detect CTCs or other rare circulating cells, such as endothelial progenitor cells (EPCs) or fetal cells, from blood, generally involve a sample preparation module (Vona G. et al., “Isolation by Size of Epithelial Tumor Cells”, American Journal of Pathology 2000, 156 (1), 57-63; S. J. Tan et al., “Microdevice for the isolation and enumeration of cancer cells from blood” Biomedical Microdevices, 2009, 11, 883-892), optionally coupled to specific staining with beads, and a flow through detector (Wang Y-N. et al, “On-chip counting the number and the percentage of CD4+ T lymphocytes”, Lab Chip 2008, 8, 309-315; Roeser T. et al., “Lab-on-chip for the Isolation and Characterization of Circulating Tumor Cells”, Proceedings of the 29th Annual International Conference of the IEEE EMBS, 2007, 6446-6448). When the cells are trapped, for example, either magnetically (Talasaz A. H. et al., “Method and apparatus for magnetic separation of cells”, WO 2009/076560; Talasaz A. H. et al., “Isolating highly enriched populations of circulating epithelial cells and other rare cells from blood using a magnetic sweeper device”, PNAS 2009, 106 (10), 3970-3975), electrically (Chen Yu et al., “Device and method for detection of analyte from a sample”, WO2010/050898) or chemically (Tang Z L. et al., “Recovery of rare cells using a microchannel apparatus with patterned posts”, US2006/0160243 A1; Nagrath S. et al., “Isolation of rare circulating tumour cells in cancer patients by microchip technology” Nature 2007, 450, 1235-1239), the cells complexes are generally not released or at the expense of their integrity. The cells are either lysed, detected optically in the same chamber (Leary J. F. et al., “Hybrid microfluidic SPR and molecular imaging device”, WO 2009/058853), or detected using a flow-through system (Soh H S. et al., “Integrated fluidics devices with magnetic sorting”, US 2008/0302732 A1).

### SUMMARY

According to an embodiment, a microfluidic system for detecting a biological entity in a sample volume is provided. The microfluidic system may include: an inlet configured to receive the sample volume; at least one microchannel in fluid communication with the inlet; a magnetic trapping region including the at least one microchannel; at least one detection region in fluid communication with the magnetic trapping region for detecting the biological entity to be detected; and at least one outlet in fluid communication with the at least one detection region.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 shows a schematic diagram of an integrated system for label-free detection of a biological entity, according to one embodiment.

FIG. 2 shows a bottom view of a fixture for mounting a microfluidic system, according to one embodiment.

FIGS. 3A and 3B show a top view and a schematic top view respectively of a microfluidic system, according to one embodiment.

FIG. 3C shows a top schematic view of a microelectrode array, according to one embodiment.

FIG. 4 shows the top views of a number of microfluidic systems, according to various embodiments.

FIGS. 5A, 5B and 5C show the schematic top views of a number of microfluidic systems of some embodiments of FIG. 4.

FIG. 6 shows a flow chart illustrating a method for detecting a biological entity in a sample volume using a microfluidic system, according to various embodiments.

FIG. 7 shows a perspective view of a microchip, according to one embodiment.

FIG. 8 shows a partial perspective view of a mold, according to one embodiment.

FIG. 9 shows a flow chart illustrating a method for manufacturing a microfluidic system, according to various embodiments.

FIG. 10 shows a schematic diagram of a system for label-free detection of a biological entity, according to one embodiment.

FIGS. 11A to 11C show fluorescence microscopy images of a trapping chamber, according to various embodiments. The scale bar in FIG. 11A represents 1 mm.

FIG. 12 shows a plot representing the sample trapping/release efficiency, according to various embodiments.

FIG. 13 shows a plot of impedance measurement and filter efficiency for some samples, according to various embodiments.

FIG. 14 shows a plot of impedance measurement, according to various embodiments.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Various embodiments provide a microfluidic system for the detection of cells, e.g. rare circulating cells such as circulating tumour cells (CTCs) for diagnosis.

Various embodiments may provide a hands-free microfluidic system and a method to integrate relatively large sample processing volume of >1 ml and up to about 30 ml, with highly sensitive label-free detection of cells, e.g. rare circulating cells such as circulating tumour cells (CTCs), in a single microfluidic package.

Various embodiments may provide a microfluidic system and a method for the detection of specific cells in low quantities (about 10 cells) in relatively large volumes (approximately >1 ml of blood), among a relatively large number of non-specific interferences (for example, >10 millions of white blood cells).

Various embodiments may provide a sample-to-answer integrated system including a microfluidic chip to detect rare circulating tumour cells (CTCs) from a relatively large blood sample volume, for cancer therapy monitoring applications. Samples may be loaded into the system and information or an answer may be projected based on the sample in a relatively short time. The integrated system may be based on a label-free impedance spectroscopy-based detection platform that may be able to detect relatively small number of cells (<10 cells). The CTCs may be isolated from other blood cells and transferred to the microfluidic chip for detection. The system may filter out other blood cells based on the size difference between the CTCs and other blood cells. The CTCs may be combined with magnetic staining using the consensual EpCAM surface marker on the CTCs. The cells may be further purified and isolated in the microfluidic chip using magnetic force. The system may be integrated with a microelectrode array for label-free highly sensitive detection. The system may provide a relatively high efficiency of purification of >80%, relatively high detection sensitivity of <10 cells and relatively high recovery efficiency (defined as the ratio of the number of cells detected over the number of cells in the sample) of 80%. The system may be able to provide processing and sensing of 3000 cells or more through the system.

Various embodiments may provide a microfluidic system and a method that may enable processing of batches of magnetically labeled cells, and e.g. magnetically labeled circulating tumour cells (CTCs), in a solution, in a single microfluidic chip that may allow label-free detection of the CTCs. The CTCs may be in a solution of diluted white blood cells, or even whole blood. The integration of batch processing and detection in a single microfluidic chip of various embodiments eliminates the need for a transferring process, thereby minimising cell loss during the sampling procedure.

Various embodiments may provide a microfluidic system and a method to transfer magnetically trapped cells from a magnetic chamber to a detection chamber where label-free detection may be performed. The transfer may be performed in a batch operation mode. Such label-free detection may employ impedance measurement as described in WO2010/050898 filed 1 Sep. 2009, which disclosure is incorporated herein by reference. In various embodiments, the sample may be purified at the magnetic trapping region by isolating the biological entity to be detected while removing any biological entity not to be detected and transferring the biological entity to be detected to a detection region. Accordingly, various embodiments may allow a reduction of the working volume at the magnetic trapping region and processing of large volumes of samples by batch processing.

In various embodiments, the cells may be loaded in a chamber on top of an electrode or a microelectrode array (MEA) coated with a specific antibody. The cells may be trapped or incubated using dielectrophoresis (DEP) for sedimentation and non-specific cells may be washed away by fluidics before detection. In various embodiments, the antibody coated on the electrode or the microelectrode array (MEA) may be specific or non-specific.

Various embodiments may provide an integrated system for detecting CTCs from blood samples. Various embodiments may provide magnetic trapping and detection of CTCs in a microfluidic system in a single microchip, packaged in a fixture with an external magnetic element, e.g. a magnet, and electrical connections to the microfluidic system and any detection systems.

Various embodiments may alleviate the challenge of sample loss during transfer between different systems or sample being lodged at interfaces between different systems by providing a microfluidic system in a single microfluidic package or a monolithic microchip which provides sample transfer from a trapping chamber to a detection chamber. The detection chamber may be based on an open-chamber concept, which may include an open chamber that may provide small sensing area for high sensitivity sensing and provide an optimal efficiency level of cell recovery through the detection process.

Various embodiments may provide a microfluidic system which advantageously does not require valves or interconnecting components between the microchannels, the magnetic trapping region and the detection region.

Various embodiments may provide a microfluidic system based on the fluidic motion or flow in the microchannels from a section of the microfluidic system to another section, thereby allowing seamless integration with label-free detection.

Various embodiments may employ an external magnetic element, for example a permanent magnet, to transfer the sample in batches to the detection region, for batch processing.

Various embodiments may provide diagnostics for cells, such as the detection of rare circulating tumour cells (CTCs), label-free detection for hands-free integrated system with high sensitivity, an automated system with processing at relatively low cost and diagnosis, prognosis and therapy monitoring, for example for cancer patients.

In various embodiments, the microfluidic system may be integrated with size filtering for blood sample processing.

In various embodiments, the microfluidic system and method may include an automated transfer between the magnetic trapping region and the detection region. The microfluidic system and method may provide detection of cells with relatively high sensitivity, relatively higher throughput and at relatively low cost. The microfluidic system may include silicon chips and plastic and metal fixtures.

In the context of various embodiments, the term “microfluidic system” may mean a fluidic system including one or more channels in the micrometer range (which may also be referred to as microchannels) where a sample volume may be provided to flow in and along the microchannels based on fluidic motion. In various embodiments, the microchannels may include a serpentile shape, a meander shape or a spiral shape. The microfluidic system may be formed on a microchip in a monolithically integrated manner to form a microfluidic chip.

In the context of various embodiments, the term “inlet” may mean an opening or a recess providing a means of entrance or intake. In various embodiments, the inlet may be connected to a microchannel or in fluid communication with a microchannel such that a sample volume provided to the inlet may flow to and through the microchannel.

In the context of various embodiments, the term “magnetic trapping region” may mean a region where a biological entity may be trapped and contained within the magnetic trapping region by an induced magnetic force acting on the magnetic trapping region. In various embodiments, the magnetic trapping region may be in fluid communication with a microchannel. An external movably arranged magnetic element, for example a magnet, may be employed to trap the biological entity in the magnetic trapping region or to release the biological entity from the magnetic trapping region, depending on the positions of the magnet. In various embodiments, the magnetic trapping region may include one or more microchannels. The microchannels may include a serpentile shape, a meander shape, a spiral shape or any combination thereof, which may increase the residence time of the biological entity, thereby allowing a relatively more efficient trapping process.

In the context of various embodiments, the term “trapping chamber” may mean a trapping region in the form of a chamber. Trapping may be by means of magnetic trapping. In various embodiments, the trapping chamber may include the magnetic trapping region. The chamber may be an open chamber or a close chamber.

In the context of various embodiments, the term “detection region” may mean a region where a biological entity may be detected. In various embodiments, the detection region may be in fluid communication with a microchannel and a magnetic trapping region. Detection may be carried out based on label-free impedance measurement or sensing. The detection region may include an electrode, a pair of electrodes or a microelectrode array including more than one electrode or more than one pair of electrodes. Each pair of electrodes may include an inner electrode and an outer electrode having a complementary shape that substantially surrounds the inner electrode. In various embodiments, the electrode, pair of electrodes or the microelectrode array may be positioned at the bottom of the detection region. In various embodiments, the biological entity may be trapped and contained within the detection region by means of dielectrophoresis or capture by antibody. The antibody may be provided or coated on the electrode or electrodes.

In the context of various embodiments, the term “detection chamber” may mean a chamber provided over the detection region. In various embodiments, the detection chamber may be based on an open-chamber concept. In various embodiments, the detection chamber may include an open chamber. In various embodiments, the electrode, pair of electrodes or the microelectrode array at the detection region may be positioned at the bottom of the detection chamber.

In the context of various embodiments, the term “open-chamber concept” may mean a concept based on or incorporating the use of an open chamber. In the context of various embodiments, the term “open chamber” may mean a chamber or a channel where a solution may flow or pass through or remain in the chamber or channel. In various embodiments, the open chamber has a top opening. In other words, the open chamber does not have a top cover.

In the context of various embodiments, the term “outlet” may mean a recess providing a structure configured to output a sample volume. In various embodiments, the outlet may be connected to a detection region or in fluid communication with a detection region such that a sample volume after the detection process may directed to the outlet to be removed from the microfluidic system. In various embodiments, the term “outlet” may also include a recess providing a structure configured to output waste. In this context, the “outlet” may be a “waste outlet”. The waste outlet may be provided between a magnetic trapping region and a detection region such that a biological entity that is not to be detected and not trapped within the magnetic trapping region during the trapping process may be directed to for removal from the microfluidic system.

In the context of various embodiments, the term “magnetic labeling device” may mean a device, apparatus or system which may provide one or more magnetic elements, for example magnetic beads, for labeling, binding or staining a biological entity. In various embodiments, the magnetic elements may include an antibody specific to the biological entity for binding the magnetic elements to the biological entity.

The term “in fluid communication” in relation to the different sections of a microfluidic system may mean a communication between two sections of the microfluidic system. In various embodiments, this communication may be a direct connection or a direct path between two sections of the microfluidic system or may include one or more intervening sections in the path between two sections of the microfluidic system.

In various embodiments, the term “biological entity” may mean a rare biomarker, a cell, an organelle, a virus particle, a biopolymer or a combination thereof. The term “cell” may include a eukaryotic cell or a prokaryotic cell. The term “cell” may also include a biomarker including a circulating tumour cell, an endothelial progenitor cell, and a fetal cell. The term “eukaryotic cell” may include a mammalian cell or a yeast cell. The term “mammalian cell” may include a tumour cell, a progenitor cell or a fetal cell.

The term “biopolymer” may include a polypeptide, a nucleic acid, a lipid and an oligosaccharide.

In various embodiments, the microchannel of the microfluidic system may have a width in the range of about 50 μm to about 500 μm, for example a range of about 50 μm to about 300 μm, a range of about 50 μm to about 200 μm, a range of about 100 μm to about 300 μm or a range of about 200 μm to about 500 μm, such that the microchannel may have a width of about 50 μm, about 100 μm, about 150 μm, about 200 μm, about 300 μm, about 400 μm or about 500 μm. In various embodiments, the microchannel may have a height in the range of about 20 μm to about 200 μm, for example a range of about 20 μm to about 100 μm, a range of about 20 μm to about 50 μm or a range of about 50 μm to about 200 μm, such that the microchannel may have a height of about 20 μm, about 40 μm, about 60 μm, about 80 μm, about 100 μm, about 150 μm or about 200 μm.

In various embodiments, the magnetic trapping region may have a dimension ranging from approximately 100 μm×100 μm to approximately 2000 μm×2000 μm (2 mm×2 mm), depending on the volume requirement of the magnetic trapping region and the strength of the magnetic force, for example a range of approximately 100 μm×100 μm to approximately 1200 μm×1200 μm, a range from approximately 100 μm×100 μm to approximately 800 μm×800 μm or a range from approximately 800 μm×800 μm to approximately 2000 μm×2000 μm, such that the magnetic trapping region may have a dimension of approximately 100 μm×100 μm, approximately 200 μm×200 μm, approximately 300 μm×300 μm, approximately 500 μm×500 μm, approximately 800 μm×800 μm, approximately 1200 μm×1200 μm or approximately 2000 μm×2000 μm.

In various embodiments, the magnetic trapping region or the trapping chamber may have a volume ranging from about 0.10 μl to about 2.0 μl, for example a range from about 0.10 μl to about 1.0 μl, a range from about 0.10 μl to about 0.50 μl or a range from about 0.50 μl to about 2.0 μl, such that the magnetic trapping region or the trapping chamber may have a volume of about 0.10 μl, about 0.15 μl, about 0.22 μl, about 0.25 μl, about 0.30 μl, about 0.37 μl, about 0.50 μl, about 0.75 μl, about 1.0 μl, about 1.2 μl, about 1.5 μl, about 1.8 μl or about 2.0 μl.

In various embodiments, the trapping flow rates or the flow rates into the magnetic trapping region or the trapping chamber may be in the range of about 10 μl/minute to about 20 μl/minute, for example a range of about 10 μl/minute to about 15 μl/minute, such that the trapping flow rate may be about 10 μl/minute, about 12 μl/minute, about 15 μl/minute, about 18 μl/minute or about 20 μl/minute

In various embodiments, the release flow rates or the flow rates out of the magnetic trapping region or the trapping chamber may be in the range of about 50 μl/minute to about 100 μl/minute, for example a range of about 50 μl/minute to about 70 μl/minute, such that the release flow rate may be about 50 μl/minute, about 60 μl/minute, about 70 μl/minute, about 80 μl/minute, about 90 μl/minute or about 100 μl/minute

In various embodiments, the detection region or the detection chamber may have a volume ranging from about 1 μl to about 10 μl, for example a range of about 1 μl to about 5 μl, such that the detection region or the detection chamber may have a volume of about 1 μl, about 1.5 μl, about 2 μl, about 2.5 μl, about 3 μl, about 3.5 μl, about 4 μl, about 5 μl, about 6 μl, about 7 μl, about 8 μl, about 9 μl or about 10 μl. In various embodiments, the volume of the detection chamber or the detection region may be approximately ten times larger than the volume of the trapping chamber or the magnetic trapping region.

In various embodiments, more than one microchannel may be provided in the microfluidic system, such that two microchannels, three microchannels or four microchannels may be provided. In various embodiments, more than one detection region may be provided, such that two detection regions, three detection regions or four detection regions may be provided. In various embodiments, more than one outlet may be provided, such that two outlets, three outlets or four outlets may be provided.

In various embodiments, the detection region may include one pair of electrodes. In further embodiments, the detection region may include a microelectrode array which may include two or more pairs of electrodes, such as two pairs of electrodes, three pairs of electrodes, four pairs of electrodes, five pairs of electrodes or six pairs of electrodes. Each pair of the electrodes may include an inner electrode and an outer electrode having a complementary shape that substantially surrounds the perimeter of the inner electrode. In various embodiments, the electrodes may be made of gold, titanium, platinum or other metals or conductive materials. In various embodiments, each of the plurality of electrodes may have a dimension smaller than 4000 μm.

In alternative embodiments, the detection region may include one electrode or a microelectrode array including two, three, four, five or six electrodes. In various embodiments, each electrode may have a dimension smaller than 4000 μm.

In various embodiments, the magnetic trapping region may include a microchannel having a serpentile shape, a meander shape, a spiral shape or any combination thereof, which may increase the residence time of the biological entity in the magnetic trapping region, where the magnetic field is relatively efficient. The shape of the microchannel may also be configured so as to lower the flow rate of the sample volume when entering the chamber, thereby lowering the shear force, while also increasing the residence time. In further embodiments, the magnetic trapping region may include a trapping chamber which may lower the flow rate of the sample volume when entering the chamber, thereby lowering the shear force, while also increasing the residence time.

In various embodiments, the microchannel in the magnetic trapping region may have the same width as the microchannel outside the magnetic trapping region. In further embodiments, the microchannel in the magnetic trapping region may have a width that is relatively larger than the width of the microchannel outside the magnetic trapping region so as to lower the flow rate of the sample volume when entering the chamber, thereby allowing easier trapping of the biological entity, lowering of the shear force, while also increasing the residence time.

In various embodiments, the microfluidic system or microfluidic chip may include a capping layer on the microfluidic system or microfluidic chip to provide adequate sealing of the microchannel. The capping layer may contact the sample containing the biological entity. The capping layer may be biocompatible. In various embodiments, the capping layer may be a polymer capping layer, made of poly(methyl methacrylate) (PMMA), polycarbonate or polydimethylsiloxane (PDMS).

In various embodiments, the sealing may be achieved based on the elastic or rubbery nature of the capping layer, which may allow the microfluidic system or microfluidic chip to be re-used.

In further embodiments, where the capping layer may be inelastic, a sealant or a gasket layer may be used to provide the sealing effect. The capping layer may be of a material sufficiently hard or heavy to provide sufficient pressure to the gasket layer to achieve sealing. The gasket layer may be a layer of polydimethylsiloxane (PDMS), silicon rubber or any biocompatible materials.

For the various embodiments, a number of design requirements were considered. A magnet or a number of magnets may be fabricated integrally on the microchip or the microfluidic system, however, the magnetic force induced by such an integral magnet may not be relatively sufficient to trap the biological entity flowing through the microchannels. Furthermore, the fabrication process may be challenging, taking into account integration with the microelectrode array.

The height of the microchannels is linked to the size of the biological entity (eg. CTCs), such that a height of about 100 μm may be provided as the biological entity may have a dimension of about 10 μm to 20 μm. The use of an external magnet may affect the choice of the dimensions of the microchannels and the flow rates. Accordingly, the design of the magnetic trapping region may be provided so as to reduce the flow rate (eg. in the order of 3 to 4 reductions) from the input microchannel into the magnetic trapping region, such that the magnetic force is relatively sufficient to trap the biological entity in the magnetic trapping region.

In order to obtain a high sensitivity at the detection region, a relatively small sensor electrode or a microelectrode array may be arranged on the detection region. In various embodiments, the sensor electrode or the microelectrode array may have one or more electrodes, each electrode having a dimension of about 100 μm. The biological entity to be detected may be trapped at the detection region. The use of dielectrophoresis (DEP) or antibody-coating or binding by antibody, alone, may in some embodiments not be sufficient to trap the biological entity as the forces involved may not cope with the speed of the biological entity as it flows along the microchannels. A detection chamber based on an open-chamber concept (ie. including an open chamber) may be provided, in conjunction with any trapping means (eg. DEP) in various embodiments. In various embodiments, the open chamber may be suitably designed to reduce the linear flow rate in a short time, enable a relatively larger concentration of the biological entity to be concentrated at the bottom of the chamber in the vicinity or on the microelectrode array and enable the DEP to more efficiently trap the biological entity.

The volume of the magnetic trapping region or the trapping chamber and the release flow rates are linked to the volume of the detection chamber or the detection region such that the volume of the detection chamber should be relatively larger to accommodate the volume of sample transferred from the trapping chamber, the volume of sample flowing between the trapping chamber and the detection chamber and the volume of fluid necessary to release the trapped biological entity. In various embodiments, the volume of the detection chamber or the detection region may be approximately ten times larger than the volume of the trapping chamber or the magnetic trapping region.

In various embodiments, a microfluidic arrangement is provided. The microfluidic arrangement may include a microfluidic system for detecting a biological entity in a sample volume, the microfluidic system including an inlet configured to receive the sample volume; at least one microchannel in fluid communication with the inlet; a magnetic trapping region including the at least one microchannel; at least one detection region in fluid communication with the magnetic trapping region for detecting the biological entity to be detected; and at least one outlet in fluid communication with the at least one detection region; and a microchip being formed on or in the microfluidic system in a monolithically integrated manner.

In various embodiments, an assembly for detecting a biological entity in a sample volume is provided. The assembly may include a microfluidic system for detecting a biological entity in a sample volume, the microfluidic system including an inlet configured to receive the sample volume; at least one microchannel in fluid communication with the inlet; a magnetic trapping region including the at least one microchannel; at least one detection region in fluid communication with the magnetic trapping region for detecting the biological entity to be detected; and at least one outlet in fluid communication with the at least one detection region; and a magnetic labeling device configured to label the biological entity to be detected with magnetic beads, wherein the magnetic labeling device is in fluid communication with the microfluidic system to supply the labeled biological entity to the microfluidic system.

In various embodiments, a method for detecting a biological entity in a sample volume using a microfluidic system for detecting a biological entity in a sample volume, the microfluidic system including an inlet configured to receive the sample volume; at least one microchannel in fluid communication with the inlet; a magnetic trapping region including the at least one microchannel; at least one detection region in fluid communication with the magnetic trapping region for detecting the biological entity to be detected; and at least one outlet in fluid communication with the at least one detection region, is provided. The method may include providing the sample volume to the at least one microchannel via the inlet; trapping the biological entity to be detected at the magnetic trapping region via a movably arranged magnet, wherein the magnetic trapping region is in fluid communication with the at least one microchannel; removing the magnet from the magnetic trapping region; transferring the biological entity from the magnetic trapping region to the at least one detection region; and detecting the biological entity in the at least one detection region.

In various embodiments, a method for manufacturing a microfluidic system is provided. The method may include providing a substrate; thinning the substrate at a predetermined portion of the substrate from a first surface of the substrate; forming a magnetic trapping region in a vicinity of the thinned portion of the substrate on a second surface of the substrate opposite the first surface; forming at least one microchannel in fluid communication with the magnetic trapping region on the substrate; forming at least one detection region in fluid communication with the magnetic trapping region; forming at least one electrode in the at least one detection region; and providing a capping layer on the substrate.

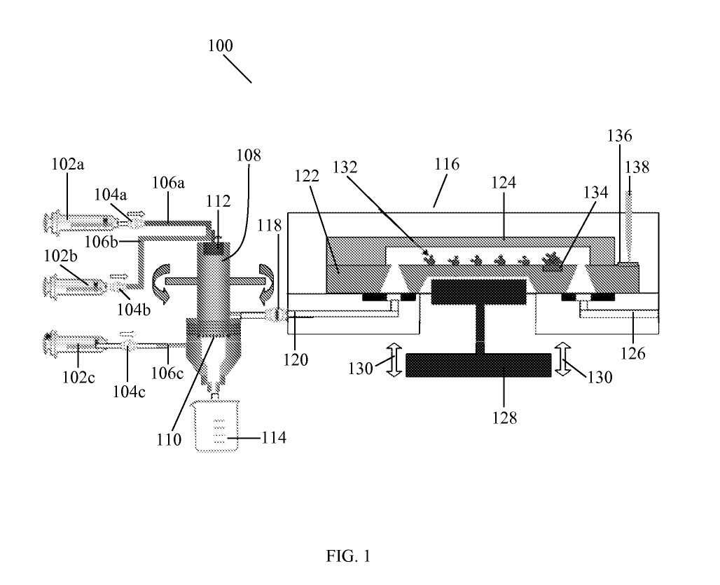

FIG. 1 shows a schematic diagram of an integrated system 100 for label-free detection of a biological entity, according to various embodiments. In various embodiments, the biological entity may be provided in a sample of blood. The integrated system 100 may include a number of supply structures, for example syringes 102a, 102b, 102c. Each of the syringes 102a, 102b, 102c, may have a corresponding valve 104a, 104b, 104c, respectively, and a corresponding interconnection, for example tubes 106a, 106b, 106c, respectively, for connections to a pump 108.

The syringes 102a, 102b, 102c, may include a biological entity or a biological sample or a buffer solution for supply to the pump 108, through the respective valves 104a, 104b, 104c, and the respective tubes 106a, 106b, 106c. In various embodiments, the pump 108 may include an integrated large area filter 110 for purification of the biological entity or the biological sample. The pump 108 may further provide an integrated magnetic labeling function for labeling of the biological entity. Waste may be generated as a result of the separation carried out through filtration by the integrated large area filter 110, which may then be collected by a waste receptacle, for example using the beaker 114, for disposal.

By way of examples and not limitations, the syringe 102a may contain a blood sample. The blood sample may contain the biological entity to be detected. The biological entity may be circulating tumour cells (CTCs). In various embodiments, the volume of the blood sample from the syringe 102a for supply to the pump 108 may be in the range from about 1 ml to about 12 ml, e.g. in the range from about 1 ml to about 8 ml, e.g. about 1 ml, about 2 ml, about 5 ml, about 8 ml, about 10 ml or about 12 ml.

The syringe 102b may contain a buffer solution. The buffer solution may be a solution of phosphate buffered saline (PBS). In various embodiments, the blood sample from the syringe 102a may be provided to the pump 108 and the PBS solution from the syringe 102b may be provided to the pump 108. During purification of the CTCs in the blood sample, the PBS solution supplied from the syringe 102b acts as an incompressible fluid to push the blood sample through the integrated large area filter 110 when the plunger 112 of the pump 108 is depressed. The PBS solution from the syringe 102b may also be provided to the pump 108 to provide a compatible environment for the blood sample containing the CTCs and as a means of keeping the pH level, the molarity and the salt concentrations of the blood sample containing the CTCs at a substantially constant value. The PBS solution may also act as a diluent to the blood sample. In various embodiments, the quantity of the PBS solution from the syringe 102b for supply to the integrated large area filter 110 of the pump 108 may be in the range from about 0.5 ml to about 10 ml, e.g. in the range from about 1 ml to about 8 ml, e.g. about 1 ml, about 2 ml, about 5 ml or about 8 ml.

The syringe 102c may contain a buffer solution. The buffer solution may be a solution of phosphate buffered saline (PBS). In various embodiments, the CTCs may be retained at the integrated large area filter 110 after filtration and may be recovered or removed from the integrated large area filter 110 by a backflow process by supplying a fluid, in a direction opposite the flow direction during the filtration process, in order to push out the CTCs retained by the integrated large area filter 110. Therefore, in various embodiments, the PBS solution supplied from the syringe 102c, may be provided to the pump 108 in a backflow process to remove the CTCs retained at the integrated large area filter 110 into the pump 108 for subsequent transfer to the microfluidic systems of various embodiments. In various embodiments, the PBS solution may also provide a compatible environment for the CTCs and also as a means of maintaining the pH level, the molarity and the salt concentrations of the sample containing the CTCs at a substantially constant value. The PBS solution may also act as a diluent. In various embodiments, the quantity of the PBS solution from the syringe 102c for supply to the integrated magnetic labeling section of the pump 108 may be in the range from about 0.5 ml to about 10 ml, e.g. in the range from about 1 ml to about 8 ml, e.g. about 1 ml, about 2 ml, about 5 ml or about 8 ml.

In various embodiments, the CTCs may be labeled or coated with magnetic elements, for example magnetic beads, that may enable the CTCs to be magnetically isolated or trapped. As conventionally known in the art, the detection of CTCs is based on the presence of the specific epithelial marker, epithelial cell adhesion molecule (EpCAM), on their surfaces. Therefore, the pump 108 may provide an integrated magnetic labeling function where the CTCs may be labeled with magnetic beads, which may be coated with an antibody specific to the EpCAM receptor. Hence, the CTCs with the magnetic elements may be isolated magnetically from other cells or constituents in the blood sample and these other cells may be separated to waste. In various embodiments, the process of labeling or coating the CTCs with magnetic beads may be carried out prior to filtration of the CTCs from the other cells or constituents of the blood sample, such that after filtration by the integrated large area filter 110, the isolated CTCs may be coated with magnetic beads, ready for transfer to the microfluidic systems of various embodiments for magnetic trapping and detection of the CTCs. For example, this may be done by diluting the blood sample with PBS and at the same time providing the magnetic beads in the solution. In further embodiments, the process of labeling or coating the CTCs with magnetic elements may be carried out after the blood sample has been filtered once and then re-suspended, for example, in the PBS solution during the backflow process. In further embodiments, the process of labeling or coating the CTCs with magnetic elements may be carried out after a number of filtration processes and backflow processes.

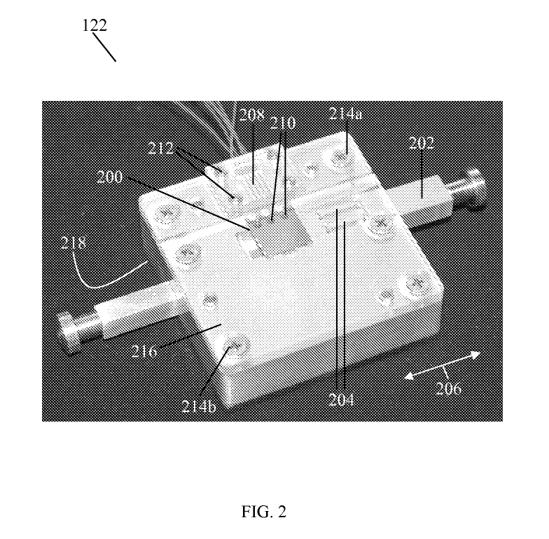

The isolated CTCs may then be provided to the assembly 116 for label-free detection of the CTCs, via the valve 118 and the inlet tube 120 to a microfluidic system. The assembly 116 may include a custom-built fixture 122 for mounting a microfluidic system or a microfluidic microchip, a microfluidic system (not shown), a capping layer 124 and an outlet tube 126 for removing the sample or waste. The custom-built fixture 122 may be of the embodiment shown in FIG. 2.

The microfluidic system may be provided for the magnetic trapping and release of the magnetically-bound CTCs for cell concentration and detection, assisted by a movably arranged magnetic element, for example the magnet 128. In various embodiments, the magnetically-bound CTCs may be trapped or released from a magnetic trapping region provided in the microfluidic system. The magnet 128 may be moved up and down relative to the fixture 122, as represented by the arrow 130. In further embodiments, the magnet 128 may be moved side to side, in and out of the magnetic trapping region. Accordingly, the magnet 128 may be provided such that the magnet in a first position may be configured to trap the magnetically-bound CTCs in the magnetic trapping region and in a second position may be configured to release the magnetically-bound CTCs.

FIG. 1 further shows magnetically-bound CTC complexes with magnetic beads, for example as represented by 132, and a detection region 134 for detecting the CTCs. The detection region 134 may include a microelectrode array for detection with a relatively high sensitivity of <10 cells. In further embodiments, the detection region 134 may provide detection sensitivity of <5 cells, <20 cells, <50 cells, <100 cells, <500 cells or <1000 cells.

The assembly 116 may further include a number of contact pads, for example 136, where probes, for example 138, may be used for electrical probing and for obtaining measurement results. The contact pads 136 may be made of gold, titanium, platinum or other metals or conductive materials.

FIG. 2 shows a bottom view of the fixture 122, having a back surface 216 and a front surface 218, for mounting a microfluidic system 200, according to various embodiments. The fixture 122 may be a custom-built fixture, fabricated using materials as known in the art for fabricating microfluidic features, for example using a metal for purposes of electrical conduction. The fixture 122 may include a movable arm 202. The movable arm 202 may include a pair of magnets 204. The movable arm 202 may be moved side to side, for example in the direction represented by the arrow 206, such that the magnets 204 may also be moved in the same direction. As a result, the magnets 204 may be moved in and out of the vicinity of the microfluidic system 200. In various embodiments, the magnets 204 may be moved in and out of the vicinity of a magnetic trapping region of the microfluidic system 200 such that the magnets 204 in a first position may trap a biological entity (eg. magnetically-bound CTCs) in the magnetic trapping region and in a second position may release the biological entity (eg. magnetically-bound CTCs). In various embodiments, the fixture 122 may include a to capping layer (not shown). The capping layer may be a polymer capping layer, made of poly(methyl methacrylate) (PMMA), polycarbonate or polydimethylsiloxane (PDMS).

The fixture 122 may further include electrical connections 208 for electrical communication with the contact pads 210 of the microfluidic system 200, which may be tightened mechanically using screws, as represented by 212 for two such screws, to provide secure electrical contacts. In various embodiments, the contact pads 210 may be made of gold. The electrical connections 208 may be connected to an electrical detection system for detection of the biological entity. The fixture 122 may further include a number of screws, as represented for example by 214a and 214b, for the purpose of tightening any attachment to be attached to the fixture 122.

In various embodiments, one magnet 204 may be provided on the movable arm 202. In further embodiments, more than two magnets 204 may be provided, such that three magnets, four magnets or five magnets may be provided.

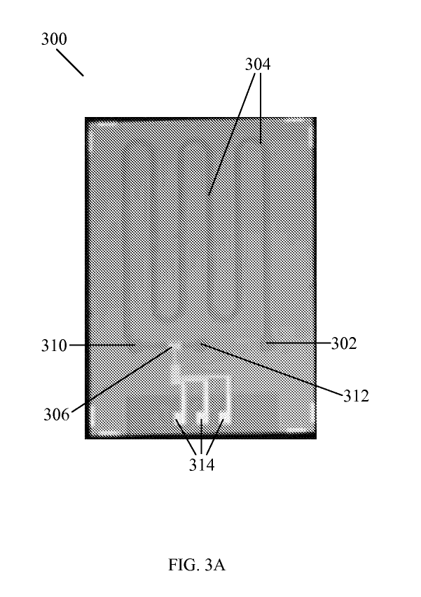

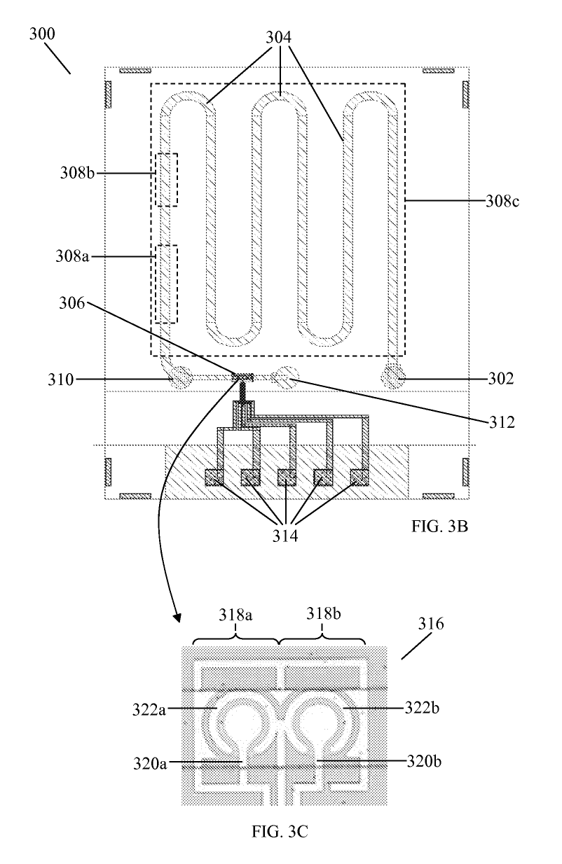

FIG. 3A and FIG. 3B show a top view and a schematic top view respectively of a microfluidic system 300, according to various embodiments. The microfluidic system 300 may be used for detecting a biological entity in a sample volume. In various embodiments, a microchip may be formed on or in the microfluidic system 300 in a monolithically integrated manner.

The microfluidic system 300 may include the inlet 302 for receiving a sample volume containing the biological entity to be detected and the microchannel 304 connected to the inlet 302. Accordingly, the microchannel 304 may be in fluid communication with the inlet 302 such that the sample volume provided to the inlet 302 may flow in and along the microchannel 304. The microchannel 304 may have a width of about 200 μm and a height of about 100 μm. In various embodiments, the microchannel 304 may have a serpentine shape, a meander shape or a spiral shape.

The microfluidic system 300 may further include a detection region 306 in fluid communication with the microchannel 304, for the detection of the biological entity to be detected. In various embodiments, the microchannel 304 may provide a magnetic trapping region for magnetically trapping the biological entity within the region, for example as represented by the dotted box 308a of FIG. 3B, such that the detection region 306 may be in fluid communication with the magnetic trapping region 308a. However, it should be appreciated that the magnetic trapping region may be of any dimension and may be provided at any portion along the microchannel 304, for example as represented by the dotted boxes 308b and 308c of FIG. 3B.

In various embodiments, the microfluidic system 300 may further include an outlet or a waste outlet 310 in fluid communication with the microchannel 304 and the magnetic trapping region 308a, 308b, 308c, where other biological entities not to be detected may be directed to. The microfluidic system 300 may further include an outlet 312 in fluid communication with the microchannel 304 and the detection region 306, for outputting the sample volume after the detection process. In various embodiments, the outlet 312 is connected to the detection region 306.

The microfluidic system 300 may further include a number of contact pads 314, shown in FIG. 3A with three contact pads 314 and in FIG. 3B with five contact pads 314. The contact pads 314 may be connected to a detection system for detection of the biological entity at the detection region 306. It should be appreciated that any number of contact pads 314 may be provided. The number of contact pads 314 provided may be based on the number of cells for detection. In various embodiments, the number of contact pads 314 may be in the range of 2 to 100, for example a range of 2 to 50 or a range of 2 to 20, such that 2 contact pads, 4 contact pads, 6 contact pads, 8 contact pads, 10 contact pads, 20 contact pads, 50 contact pads or 100 contact pads, may be provided. In various embodiments, the contact pads 314 may function as a working contact pad, a counter contact pad or a reference contact pad. In various embodiments, at least two contact pads 314 are provided with one contact pad functioning as the working contact pad and the other contact pad functioning collectively as the counter and reference contact pad. In various embodiments with more than two contact pads, any number of contact pads may function as the working contact pads, the counter contact pads, the reference contact pads or the collective counter/reference contact pads. In various embodiments, the contact pads 314 may be made of gold.

In various embodiments, the detection region 306 may include a microelectrode array 316 as shown in FIG. 3C. The microelectrode array 316 may be in electrical communication with the contact pads 314 such that the microfluidic system 300 or the microfluidic chip may be in electrical communication with a detection system for detection of the biological entity. In various embodiments, the detection region 306 may be provided within a detection chamber. The detection chamber may be an open chamber.

In various embodiments, the microfluidic system 300 may include a capping layer (not shown) to provide adequate sealing of the microchannel 304 and the magnetic trapping region 308a, 308b, 308c. The capping layer may be a polydimethylsiloxane (PDMS) capping layer. The capping layer may be biocompatible. The PDMS capping layer may be casted on a mold containing a number of patterns or structures which may allow for the formation of openings on the capping layer, corresponding to the sample inlet and outlets, as well as the formation of a detection chamber over the detection region 306 including the microelectrode array.

In various embodiments, the microfluidic system 300 may be positioned over a movably arranged magnetic element, for example a magnet. The magnet may be provided in the vicinity of the microchannel 304 and the magnetic trapping region 308a, 308b, 308c to magnetically trap the biological entity to be detected within the magnetic trapping region 308a, 308b, 308c, or to release the biological entity to the detection region 306 for detection of the biological entity. In various embodiments, batch processing may be performed such that in each batch, a sample volume of about 10 μl to about 100 μl, e.g. about 10 μl to about 20 μl, about 10 μl to about 50 μl or about 50 μl to about 100 μl, may be processed.

FIG. 3C shows a top schematic view of a microelectrode array 316, according to various embodiments. The microelectrode array 316 includes two pairs of electrodes 318a, 318b. Each pair of electrodes 318a, 318b, may include, respectively, an inner electrode 320a, 320b and an outer electrode 322a, 322b. The outer electrodes 322a, 322b may substantially surround its corresponding inner electrodes 320a, 320b.

In the embodiment of FIG. 3C, each of the inner electrodes 320a, 320b includes a disc electrode while each of the outer electrodes 322a, 322b includes a horseshoe electrode. However, it should be appreciated that the inner electrodes 320a, 320b may be of any shape, for example a triangular shape, an oval shape, a square shape, a rectangular shape or a diamond shape and the outer electrodes 322a, 322b may be in the form of a narrow strip having a complementary shape that substantially surrounds the perimeter of the corresponding inner electrodes 320a, 320b.

In various embodiments, each pair of electrodes 318a, 318b may be coated with a capture molecule, for example a cell specific antibody, that recognizes and binds the biological entity to be identified, detected or quantified. In various embodiments, the capture molecule may be a protein, an antibody, a ligand, a receptor, an inhibitor, a small molecule, a nucleic acid molecule, a hormone or a non-cleavable substrate analogue.

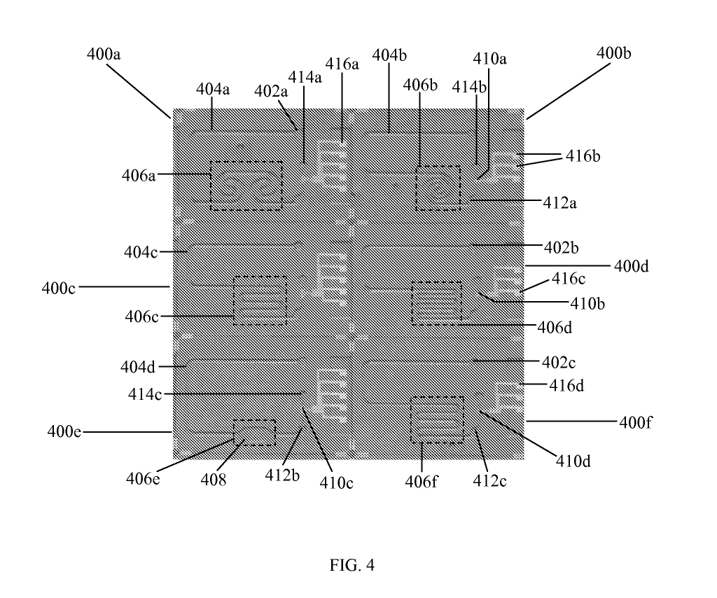

FIG. 4 shows the top views of a number of microfluidic systems 400a, 400b, 400c, 400d, 400e, 400f, according various embodiments. The microfluidic systems 400a, 400b, 400c, 400d, 400e, 400f, may be used for detecting a biological entity in a sample volume. In various embodiments, a microchip may be formed on or in each of the microfluidic systems 400a, 400b, 400c, 400d, 400e, 400f, in a monolithically integrated manner.

Each of the microfluidic systems 400a, 400b, 400c, 400d, 400e, 400f, may include an inlet, for example 402a, 402b, 402c, for receiving a sample volume containing the biological entity to be detected and a microchannel, for example 404a, 404b, 404c, 404d, connected to the respective inlet. Accordingly, each of the microchannels, for example 404a, 404b, 404c, 404d, of the microfluidic systems 400a, 400b, 400c, 400d, 400e, 400f, may be in fluid communication with its respective inlet such that the sample volume provided to the inlet may flow in and along its respective microchannel. The microchannels, for example 404a, 404b, 404c, 404d, may have a width of about 200 μm and a height of about 100 μm. In various embodiments of FIG. 4, the microchannels, for example 404a, 404b, 404c, 404d, may include a serpentine shape, a meander shape, a spiral shape or any combination thereof.

Each of the microfluidic systems 400a, 400b, 400c, 400d, 400e, 400f, may further include a magnetic trapping region, for example 406a, 406b, 406c, 406d, 406e, 406f, for magnetically trapping the biological entity within the region. Each of the magnetic trapping regions of the microfluidic systems 400a, 400b, 400c, 400d, 400e, 400f, may include a microchannel. In various embodiments, the microchannel within the magnetic trapping region may include a serpentine shape, a meander shape, a spiral shape or any combination thereof In various embodiments, the magnetic trapping region may be provided within a respective trapping chamber. In alternative embodiments, the magnetic trapping region may include a trapping chamber 408, as shown in the embodiment of microfluidic system 400e.

Each of the microfluidic systems 400a, 400b, 400c, 400d, 400e, 400f, may further include a detection region, for example 410a, 410b, 410c, 410d, in fluid communication with its respective magnetic trapping region, for the detection of the biological entity. In various embodiments, the detection regions, for example 410a, 410b, 410c, 410d, may be provided within a respective detection chamber. The detection chamber may be an open chamber.

In various embodiments, each of the microfluidic systems 400a, 400b, 400c, 400d, 400e, 400f, may further include an outlet or a waste outlet, for example 412a, 412b, 412c, in fluid communication with its respective microchannel and respective magnetic trapping region, where other biological entities not to be detected may be directed to. The waste outlet, for example 412a, 412b, 412c, may be arranged between the detection region, for example 410a, 410b, 410c, 410d, and the magnetic trapping region, for example 406a, 406b, 406c, 406d, 406e, 406f. Each of the microfluidic systems 400a, 400b, 400c, 400d, 400e, 400f, may further include an outlet, for example 414a, 414b, 414c, in fluid communication with its respective microchannel and respective detection region, for outputting the sample volume.

In various embodiments, each of the microfluidic systems 400a, 400b, 400c, 400d, 400e, 400f, may further include a number of contact pads, for example 416a, 416b, 416c, 416d. The contact pads, for example 416a, 416b, 416c, 416d, may be connected to a corresponding detection system for detection of the biological entity at the corresponding detection region. It should be appreciated that any number of contact pads may be provided, such that two contact pads, three contact pads, four contact pads, two contact pads or six contact pads may be provided. In various embodiments, the contact pads, for example 416a, 416b, 416c, 416d, may be made of gold.

In various embodiments, the detection region, for example 410a, 410b, 410c, 410d, may include a microelectrode array, for example based on the embodiment shown in FIG. 3C. The microelectrode array may be in electrical communication with the contact pads, for example 416a, 416b, 416c, 416d, such that each of the microfluidic systems 400a, 400b, 400c, 400d, 400e, 400f, or the corresponding microchip may be in electrical communication with a corresponding detection system for detection of the biological entity.

In various embodiments, the microfluidic systems 400a, 400b, 400c, 400d, 400e, 400f may include a capping layer (not shown) to provide adequate sealing of the respective microchannels and the respective magnetic trapping regions. The capping layer may be biocompatible. The capping layer may be a polydimethylsiloxane (PDMS) capping layer. The PDMS capping layer may be casted on a mold containing a number of patterns or structures which may allow for the formation of openings on the capping layer corresponding to the sample inlet and outlets, as well as the formation of a detection chamber over the detection region including the microelectrode array.

In various embodiments, each of the microfluidic systems 400a, 400b, 400c, 400d, 400e, 400f, may be positioned over a movably arranged magnetic element, for example a magnet. The magnet may be provided in the vicinity of the magnetic trapping region, for example 406a, 406b, 406c, 406d, 406e, 406f, to magnetically trap the biological entity to be detected in the magnetic trapping region, for example 406a, 406b, 406c, 406d, 406e, 406f, or to release the biological entity from the magnetic trapping region, for example 406a, 406b, 406c, 406d, 406e, 406f, to the detection regions, for example 410a, 410b, 410c, 410d, for detection of the biological entity. In various embodiments, batch processing may be performed such that in each batch, a sample volume of about 10 μl to about 100 μl, e.g. about 10 μl to about 20 μl, about 10 μl to about 50 μl or about 50 μl to about 100 μl, may be trapped and released.

It should be appreciated that while the microfluidic systems 400a, 400b, 400c, 400d, 400e, 400f, have been described using only a selected number of examples for the inlet, microchannel, magnetic trapping region, detection region, waste outlet, outlet and contact pads, each of the microfluidic systems 400a, 400b, 400c, 400d, 400e, 400f, may have a corresponding inlet, a corresponding microchannel, a corresponding magnetic trapping region, a corresponding detection region, a corresponding waste outlet, a corresponding outlet and a number of corresponding contact pads.

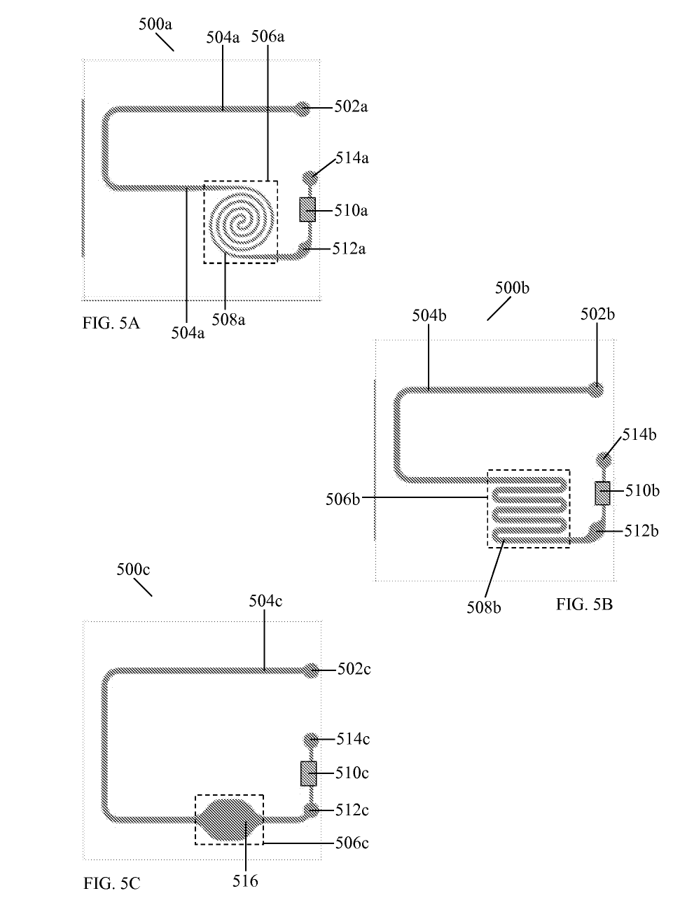

FIG. 5A, FIG. 5B and FIG. 5C show the schematic top views of microfluidic systems 500a, 500b, 500c, respectively. The microfluidic systems 500a, 500b, 500c, may be used for detecting a biological entity in a sample volume. In various embodiments, a microchip may be formed on or in each of the microfluidic systems 500a, 500b, 500c, in a monolithically integrated manner. The schematic views of the microfluidic systems 500a, 500b, 500c correspond to the embodiments of the microfluidic systems 400b, 400d, 400e respectively, of FIG. 4.

In FIG. 5A, the microfluidic system 500a may include the inlet 502a for receiving a sample volume containing the biological entity to be detected and the microchannel 504a connected to the inlet 502a. Accordingly, the microchannel 504a may be in fluid communication with the inlet 502a such that the sample volume provided to the inlet 502a may flow in and along the microchannel 504a. The microchannel 504a may have a meander shape. The microchannel 504a may have a width of about 200 μm and a height of about 100 μm.

The microfluidic system 500a may further include a magnetic trapping region 506a for magnetically trapping the biological entity within the region. The magnetic trapping region 506a may include the microchannel 508a such that the magnetic trapping region 506a and the microchannel 508a within the magnetic trapping region 506a may be in fluid communication with the microchannel 504a located outside of the magnetic trapping region 506a. The microchannel 508a may include a spiral shape that may increase the residence time of the biological entity in the magnetic trapping region 506a. The volume of the magnetic trapping region 506a may be about 0.22 μl. In various embodiments, the magnetic trapping region 506a may be provided within a trapping chamber.

In various embodiments, the microchannel 508a in the magnetic trapping region 506a may have the same width as the microchannel 504a outside the magnetic trapping region 506a. In further embodiments, the microchannel 508a in the magnetic trapping region 506a may have a width that is relatively larger than the width of the microchannel 504a outside the magnetic trapping region 506a.

The microfluidic system 500a may further include a detection region 510a in fluid communication with the magnetic trapping region 506a and the microchannels 504a, 508a, for the detection of the biological entity to be detected. In various embodiments, the detection region 510a may be provided within a detection chamber. The detection chamber may be an open chamber.

In various embodiments, the microfluidic system 500a may further include a waste outlet 512a in fluid communication with the microchannels 504a, 508a, the magnetic trapping region 506a and the detection region 510a, where biological entities not to be detected may be directed to. The microfluidic system 500a may further include an outlet 514a in fluid communication with the microchannels 504a, 508a and the detection region 510a, for outputting the sample volume.

In FIG. 5B, the microfluidic system 500b may include the inlet 502b for receiving a sample volume containing the biological entity to be detected and the microchannel 504b connected to the inlet 502b. Accordingly, the microchannel 504b may be in fluid communication with the inlet 502b such that the sample volume provided to the inlet 502b may flow in and along the microchannel 504b. The microchannel 504b may have a meander shape. The microchannel 504b may have a width of about 200 μm and a height of about 100 μm.

The microfluidic system 500b may further include a magnetic trapping region 506b for magnetically trapping the biological entity within the region. The magnetic trapping region 506b may include the microchannel 508b such that the magnetic trapping region 506b and the microchannel 508b within the magnetic trapping region 506b may be in fluid communication with the microchannel 504b located outside of the magnetic trapping region 506b. The microchannel 508b may include a meander shape that increases the residence time of the biological entity in the magnetic trapping region 506b. The volume of the magnetic trapping region 506b may be about 0.25 μl. In various embodiments, the magnetic trapping region 506b may be provided within a trapping chamber.

In various embodiments, the microchannel 508b in the magnetic trapping region 506b may have the same width as the microchannel 504b outside the magnetic trapping region 506b. In further embodiments, the microchannel 508b in the magnetic trapping region 506b may have a width that is relatively larger than the width of the microchannel 504b outside the magnetic trapping region 506b.

The microfluidic system 500b may further include a detection region 510b in fluid communication with the magnetic trapping region 506b and the microchannels 504b, 508b, for the detection of the biological entity to be detected. In various embodiments, the detection region 510b may be provided within a detection chamber. The detection chamber may be an open chamber.

In various embodiments, the microfluidic system 500b may further include a waste outlet 512b in fluid communication with the microchannels 504b, 508b, the magnetic trapping region 506b and the detection region 510b, where biological entities not to be detected may be directed to. The microfluidic system 500b may further include an outlet 514b in fluid communication with the microchannels 504b, 508b and the detection region 510b, for outputting the sample volume.

In FIG. 5C, the microfluidic system 500c may include the inlet 502c for receiving a sample volume containing the biological entity to be detected and the microchannel 504c connected to the inlet 502c. Accordingly, the microchannel 504c may be in fluid communication with the inlet 502c such that the sample volume provided to the inlet 502c may flow in and along the microchannel 504c. The microchannel 504c may have a meander shape. The microchannel 504c may have a width of about 200 μm and a height of about 100 μm.

The microfluidic system 500c may further include a magnetic trapping region 506c for magnetically trapping the biological entity within the region. The magnetic trapping region 506c may include a trapping chamber 516 such that the magnetic trapping region 506c and the trapping chamber 516 may be in fluid communication with the microchannel 504c. The trapping chamber 516 may have a volume of about 0.37 μl. The trapping chamber 516 may lower the flow rate of the sample volume when entering the trapping chamber 516, thereby lowering the shear force, while also increasing the residence time.

The microfluidic system 500c may further include a detection region 510c in fluid communication with the magnetic trapping region 506c and the microchannel 504c for the detection of the biological entity to be detected. In various embodiments, the detection region 510c may be provided within a detection chamber. The detection chamber may be an open chamber.

In various embodiments, the microfluidic system 500c may further include a waste outlet 512c in fluid communication with the microchannel 504c, the magnetic trapping region 506c and the detection region 510c, where biological entities not to be detected may be directed to. The microfluidic system 500c may further include an outlet 514c in fluid communication with the microchannel 504c and the detection region 510c, for outputting the sample volume.

In various embodiments, the detection regions 510a, 510b, 510c may include a microelectrode array (not shown). The microfluidic systems 500a, 500b, 500c, may further include a number of contact pads (not shown) in electrical communication with the microelectrode arrays of the respective detection regions, 510a, 510b, 510c, and a corresponding detection system for the detection of the biological entity at the detection regions 510a, 510b, 510c. In various embodiments, three or five contact pads may be provided.

In various embodiments, the microfluidic systems 500a, 500b, 500c, may include a capping layer (not shown) to provide adequate sealing of the microchannels 504a, 504b, 504c, 508a, 508b, and the magnetic trapping region 506a, 506b, 506c. The capping layer may be biocompatible. The capping layer may be a polydimethylsiloxane (PDMS) capping layer. The PDMS capping layer may be casted on a mold containing a number of patterns or structures which may allow for the formation of openings on the capping layer, corresponding to the sample inlet and outlets, as well as the formation of a detection chamber over the detection region 510a, 510b, 510c, including the microelectrode array.

In various embodiments, the microfluidic systems 500a, 500b, 500c, may be positioned over a movably arranged magnetic element, for example a magnet. The magnet may be provided in the vicinity of the magnetic trapping region 506a, 506b, 506c, to magnetically trap the biological entity to be detected or to release the biological entity to the detection regions 510a, 510b, 510c. In various embodiments, batch processing may be performed such that in each batch, a sample volume of about 10 μl to about 100 μl, e.g. about 10 μl to about 20 μl, about 10 μl to about 50 μl or about 50 μl to about 100 μl, may be trapped and released.

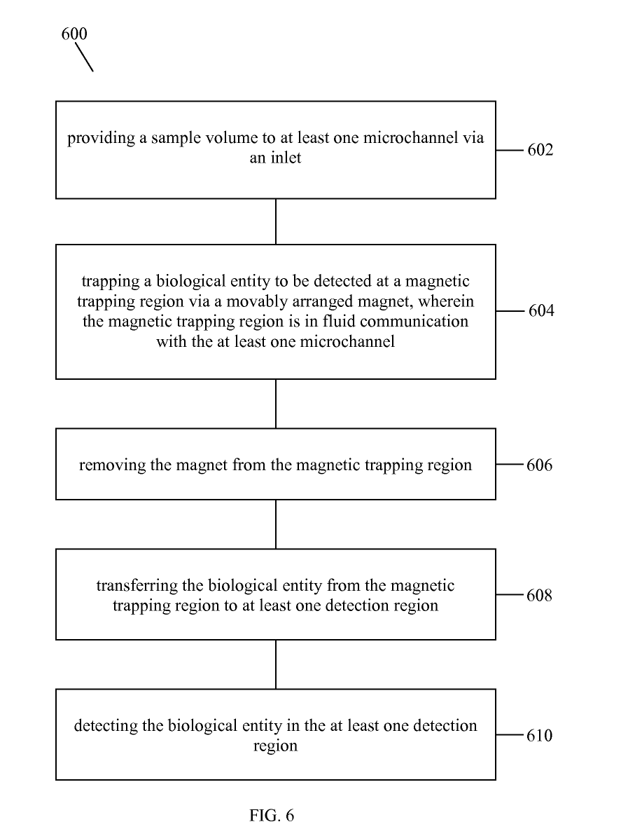

FIG. 6 shows a flow chart 600 illustrating a method for detecting a biological entity in a sample volume using a microfluidic system, according to various embodiments.

At 602, a sample volume is provided to at least one microchannel via an inlet.

At 604, a biological entity to be detected is trapped at a magnetic trapping region via a movably arranged magnet, wherein the magnetic trapping region is in fluid communication with the at least one microchannel.

At 606, the magnet is removed from the magnetic trapping region.

At 608, the biological entity is transferred from the magnetic trapping region to at least one detection region.

At 610, the biological entity is detected in the at least one detection region.

The processing of the sample and the detection of the biological entity will now be described, by way of examples and not limitations, based on a blood sample containing negative white blood cells and circulating tumour cells (CTCs). The negative white blood cells are not labeled with magnetic beads while the CTCs, as the biological entity to be detected, are labeled with magnetic beads.

The sample is provided into the microchannels via the inlet, for example using a pump with tubing connected to the inlet. The blood sample containing the cells subsequently flows along the microchannels.

A movably arranged magnet is positioned in the vicinity and close to the magnetic trapping region. As the blood sample flows through the magnetic trapping region, the CTCs with the magnetic beads are trapped at the magnetic trapping region, while the white blood cells without magnetic beads are evacuated to the waste through the waste outlet.

A batch having a volume of about 10 μl to about 100 μl is trapped and the magnet is then removed from the vicinity of the magnetic trapping region to release the CTCs. The batch sample is transferred to the detection chamber in the detection region to fill the detection chamber. In various embodiments, the magnet may be removed from the vicinity of the magnetic trapping region after a predefined amount of the sample has passed the magnetic trapping region.

Washing may be carried out before detection to remove any negative white blood cells that may not have been directed to waste and which are transferred to the detection chamber. The CTCs are trapped in the detection chamber, for example using dielectrophoresis (DEP) or capture and binding by antibody. Detection of the CTCs is then carried out, for example based on impedance measurements. Alternatively, the batch sample is kept in the detection chamber while awaiting the transfer of a second batch sample or subsequent batch samples into the detection chamber.

After the detection process, the content of the detection chamber is removed through the outlet so that the next batch sample may be processed and detected.

### Fabrication and Experimental Data

The fabrication of the microfluidic system of various embodiments will now be described as follows, by way of examples and not limitations.

### Microchip Design and Fabrication

A silicon microchip was fabricated using conventional silicon fabrication processes. A silicon substrate was first prepared. The silicon substrate may be approximately 12 mm×12 mm×750 μm. A predetermined portion of the substrate on the back surface of the substrate was then etched using potassium hydroxide (KOH) to thin down the silicon substrate to approximately 300 μm to create a depression.

An SU8 photoresist was spin-coated to deposit a layer of photoresist having a thickness of about 100 μm on the front surface of the silicon substrate. Selective patterning was then performed using conventional lithography processes to define the microchannels, the magnetic trapping region or the trapping chamber, the detection region, the inlet, the outlet and the waste outlet. Accordingly, the bottom surface of the microchannels is formed on the silicon substrate of the microchip.

The magnetic trapping region or the trapping chamber was patterned and provided in the vicinity of the thinned portion of the substrate so that a magnet may be arranged as close as possible to the flowing sample so as to provide an efficient trapping force.

Metal electrodes and contact pads were then fabricated based on metal deposition and patterning. Gold (Au) and titanium (Ti) were deposited on the surface of the silicon substrate corresponding to the position of the detection region and selectively patterned to form the electrode or the microelectrode array. Gold and Ti were also used for the fabrication of the contact pads.

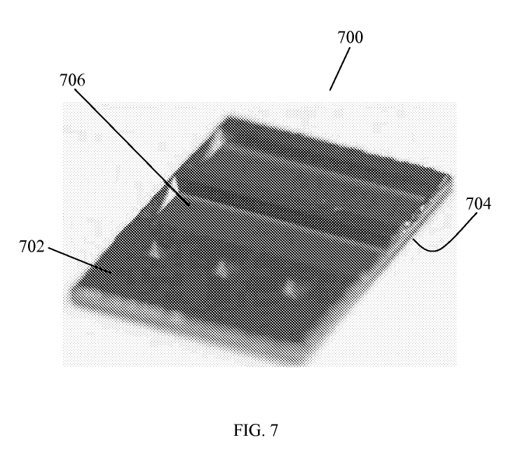

FIG. 7 shows a perspective view of a microchip 700 having a back surface 702 and a front surface 704, fabricated according to various embodiments. The fabricated microchip 700 includes a depression 706 on the back surface 702.

### PDMS Capping Layer Design and Fabrication

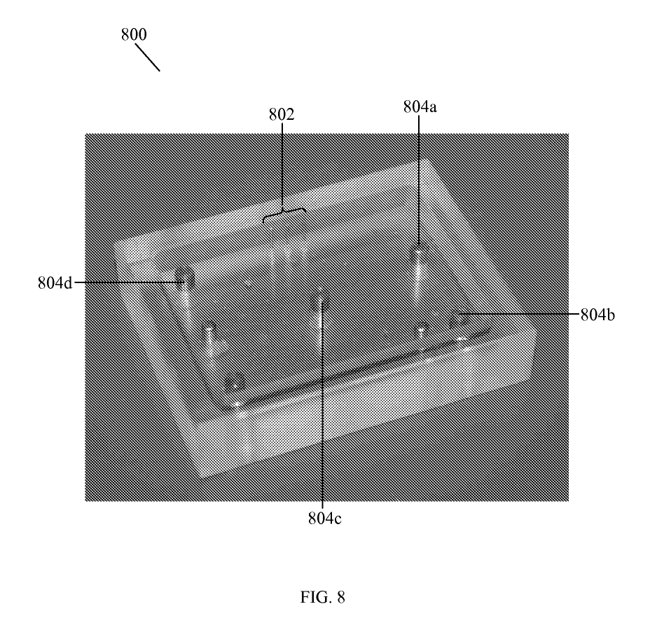

A polydimethylsiloxane (PDMS) capping layer having a thickness of about 10 mm was provided to cap the microchip. The PDMS capping layer may be biocompatible. The PDMS capping layer was molded using a custom designed mold. FIG. 8 shows a partial perspective view of the mold 800 according to various embodiments, for molding the PDMS capping layer.

The mold 800 includes pins 802 with a diameter of 0.6 mm, arranged to position the pins 802 at locations corresponding to the inlet, the outlet, the waste outlet and the detection region of the microchip, in order to create openings in the capping layer. The mold 800 further includes a number of cylindrical structures, for example 804a, 804b, 804c, 804d, for molding a complementary structure on the PDMS capping layer.

A detection chamber at the detection region was designed using a square piece having the dimensions of approximately 300 μm×500 μm×2000 μm, obtained via plastic machining, and placed in contact with the pin corresponding to the detection region of the microchip.

Subsequently, a mixture of PDMS primer was poured into the mold and the mixture cured at room temperature for a day to minimize shrinkage.

The microchip including the microfluidic system may be positioned in a fixture for mounting the microchip, such as the embodiment shown in FIG. 2. The microchip may be positioned in the location of the microfluidic system 200 of FIG. 2. Based on the embodiment of FIG. 2, the cured PDMS capping layer was then positioned on top of the microchip and also over the fixture 122 (FIG. 2) on the surface 218 (FIG. 2) to seal the microchip and its microchannels. Accordingly, the PDMS capping layer forms the ceiling of the microchannels. The PDMS capping layer is attached to the fixture 122 (FIG. 2) by means of the tightening screws, as represented for example by 214a and 214b corresponding to the positions of the complementary structures on the capping layer formed by the cylindrical structures, for example 804a, 804b, 804c, 804d, of the mold 800.

In various embodiments, the microfluidic system, the microchip and the PDMS capping layer may be disposable. The custom-built fixture for mounting the microfluidic chip or microfluidic system may be re-used.

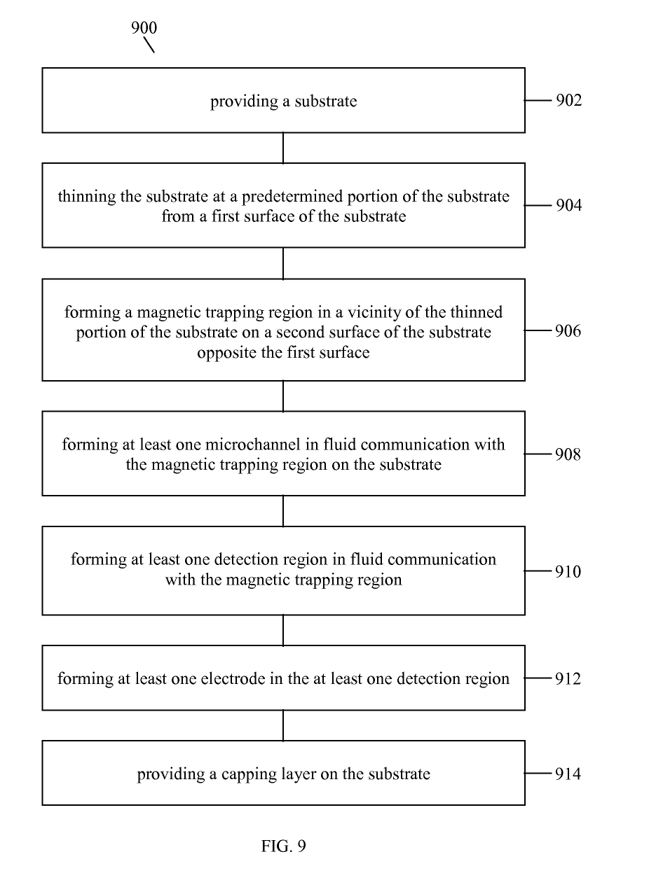

FIG. 9 shows a flow chart 900 illustrating a method for manufacturing a microfluidic system, according to various embodiments.

At 902, a substrate is provided.

At 904, the substrate is thinned at a predetermined portion of the substrate from a first surface of the substrate.

At 906, a magnetic trapping region is formed in a vicinity of the thinned portion of the substrate on a second surface of the substrate opposite the first surface.

At 908, at least one microchannel is formed in fluid communication with the magnetic trapping region on the substrate.

At 910, at least one detection region is formed in fluid communication with the magnetic trapping region.

At 912, at least one electrode is formed in the at least one detection region.

At 914, a capping layer is provided on the substrate.

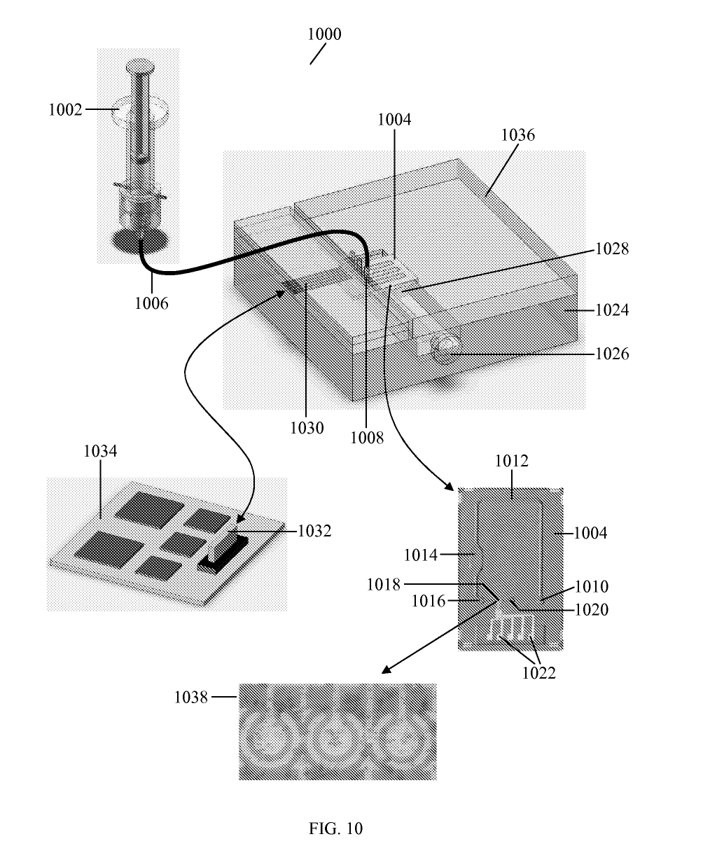

FIG. 10 shows a schematic diagram of a system 1000 for label-free detection of a biological entity, according to various embodiments. The system 1000 may include a syringe or a pump 1002 which may hold a relatively large sample volume. By way of examples, a 8 ml blood sample containing red blood cells, white blood cells and circulating tumour cells (CTCs) may be provided to the pump 1002. The pump 1002 may include an integrated filter for size filtration and purification to separate the red blood cells and the white blood cells from the relatively bigger CTCs. After purification, the blood sample containing the CTCs may be supplied to an integrated magnetic labeling section of the pump 1002 for binding with magnetic elements, for example magnetic beads, which may be coated with an antibody specific to the EpCAM receptor on the CTCs.

The blood sample containing the CTCs may then be provided to the microfluidic chip 1004 via a tubing 1006 connected between the pump 1002 and the pin 1008 connecting to the inlet 1010 of the microfluidic chip 1004. In various embodiments, the microfluidic chip 1004 includes a microfluidic system which may include a microchannel 1012, a trapping chamber 1014, a waste outlet 1016, a detection region 1018, an outlet 1020 and a number of contact pads, as represented by 1022. The detection region 1018 may include a detection chamber. The detection chamber may be an open chamber.

In various embodiment, the microfluidic chip 1004 may be mounted on a fixture 1024. The fixture 1024 may include a movable arm 1026. The movable arm 1026 may include a pair of magnets, for example as represented by 1028. The movable arm 1026 may be moved in and out of position beneath the microfluidic chip 1004. As a result, the magnets 1028 may be moved in and out of the vicinity of the trapping chamber 1014 such that the magnets 1028 may induce a magnetic force to trap the magnetically-bound CTCs in the trapping chamber 1014 when the magnets 1028 are positioned adjacently under the microfluidic chip 1004 and may release the magnetically-bound CTCs when the magnets 1028 are moved away from the microfluidic chip 1004. During the trapping of the magnetically-bound CTCs, the red blood cells and the white blood cells that may be present in the blood sample may be removed to the waste outlet 1016.

In various embodiments, the fixture 1024 may further include a number of electrical connections 1030 for electrical communications with the contact pads 1022 of the microfluidic chip 1004 and the connector 1032 of a printed circuit board (PCB) 1034 for electrical control of the microfluidic chip 1004. In various embodiments, a capping layer 1036 may be provided on top of the microfluidic chip 1004 and the fixture 1024 to seal the microfluidic chip 1004 and the microchannel 1012.

In various embodiments, the detection region 1018 of the microfluidic chip 1004 may include a microelectrode array 1038.

### Sample Loading, Magnetic Trapping and Detection

Metal pins (22 gauge) and tubings were fitted in the openings in the PDMS capping layer at positions corresponding to the inlet, the outlet and the waste outlet. The other ends of the tubings were connected to syringe pumps or to waste collection.

For characterization of the microchip prior to any measurements, a mock blood sample containing a concentration of approximately 5 million/ml of T-lymphocytes from a cell line (Jurkat), spiked with about 10-200 breast cancer cells in a PBS solution with approximately 10% glycerol to mimic blood viscosity, was used.

For measurement purposes, the tubing to be connected to the inlet was filled with a buffer solution of phosphate buffered saline (PBS). A 10 μl sample containing 1343 MCF7 breast cancer cells (+/−156) labeled with 3.28 μm magnetic beads (in conformity with the supplier's protocol with about 3-4 magnetic beads per cell) and a red colored fluorescent dye (wheat germ agglutinin by Invitrogen) were drawn manually into the tubing, followed by 20 μl of PBS, to minimize the drawing in of air resulting in air bubbles in the tubing.

The tubing was then connected to the inlet and about 20 μl of the sample was flowed through the microchannels at a flow rate of about 150 μl/min to prevent the cells from settling in the tubing. This corresponds to the additional volume of PBS drawn in the tubing at the previous stage, where no significant diffusion of the cells was observed. The flow rate was then decreased to about 10 μl/min so that the cells were trapped at the magnetic trapping region or the trapping chamber by the use of a magnet positioned below the microchip, as illustrated in FIG. 10. The magnet was then removed and the flow rate increased to about 25 μl/min to release the cells. A total of about 300 μl of the cell sample was flowed through the microchannels and to waste during the operation.

To establish the efficiency of the procedure, the cells present in the waste after the trapping process were counted under a microscope, after undergoing a conventional filtration process employing a membrane or a filter having a pore size of about 5 μm. The efficiency of the filtration process was approximately 90%, meaning that 90% of the cells are retained on the filter, while 10% may be lost. The cell count was approximately 226±58. Such a counting method yields a precision of about 15%±9%.

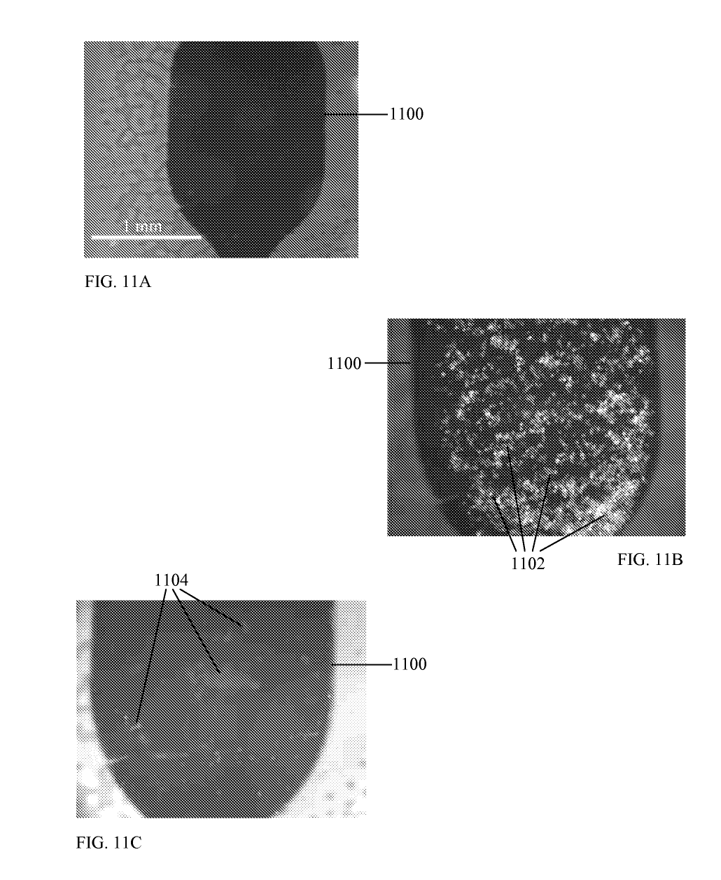

FIGS. 11A to 11C show fluorescence microscopy images of a trapping chamber, according to various embodiments. FIG. 11A shows a trapping chamber 1100 that is empty, prior to the trapping of cells in the trapping chamber 1100. FIG. 11B shows the trapping chamber 1100 after a trapping operation with a magnet positioned in the vicinity of the trapping chamber 1100, and assisted by a decrease in the sample flow rate. As shown in FIG. 11B, MCF7 cells 1102 bound with magnetic beads are trapped in the trapping chamber 1100. FIG. 11C shows the trapping chamber 1100 after a release operation with the magnet removed from the vicinity of the trapping chamber 1100, and assisted by an increase in the sample flow rate. As shown in FIG. 11C, the trapping chamber 1100 is empty, except for a minimal amount of residual MCF7 cells 1104.

For measurements purposes, a sample of approximately 500 μl of blood spiked with about 10-175 MCF7 breast cancer cells was prepared. The sample was then filtered and approximately 10 μl of the filtered sample was processed according to various embodiments, for example magnetically labelling the cells. The sample was subsequently processed in the microfluidic system of various embodiments. The cells were trapped and then released for transfer to the detection chamber, based on the open-chamber concept. The trapping flow rate was approximately 10 μl/min and the release flow rate was approximately 25 μl/min. Prior to the release of the cells from the trapping chamber, an impedance measurement in buffer was performed. Subsequently, the buffer was removed from the trapping chamber. Upon release, the cells were transferred to the detection chamber, by flowing about 2 μl of the sample at a flow rate of about 25 μl/min. At the detection chamber, the cells were incubated using dielectrophoresis (DEP) at a voltage of about 0.6 V and a frequency of about 2 MHz for about 10 minutes on the antibody-coated microelectrode array in the detection chamber that binds the cells. The non-specific cells to the antibody were washed away at a flow rate of about 25 μl/min and a second impedance measurement was performed. Alternatively, a second batch of sample may be transferred from the magnetic trapping chamber to the detection chamber.

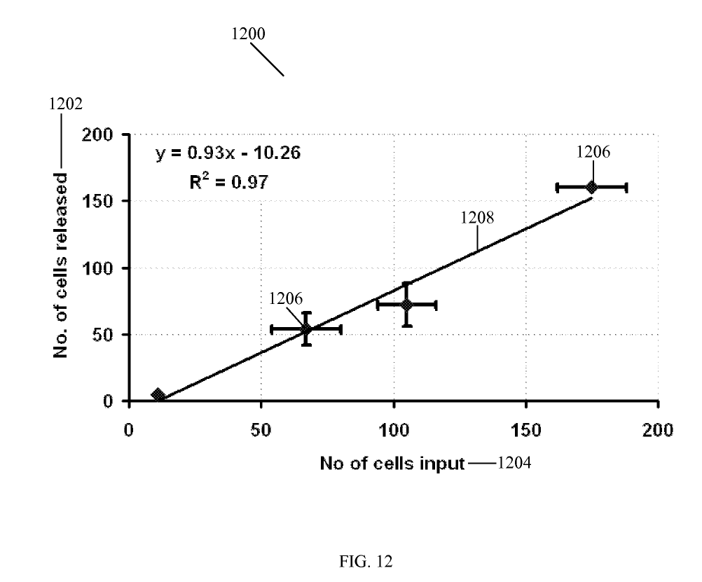

FIG. 12 shows a plot 1200 representing the sample trapping/release efficiency, as represented by the data points 1206, according to various embodiments. The plot 1200 is shown in terms of the number of cells released 1202 from the magnetic trapping region against the number of cells input 1204. The line 1208 represents a linear fit through the data points 1206 and may be indicated as a linear relationship having the empirical equation y=0.93x−10.26 and a square of the correlation coefficient, R², having a value of 0.97, indicating relatively good linear reliability of the linear relationship.

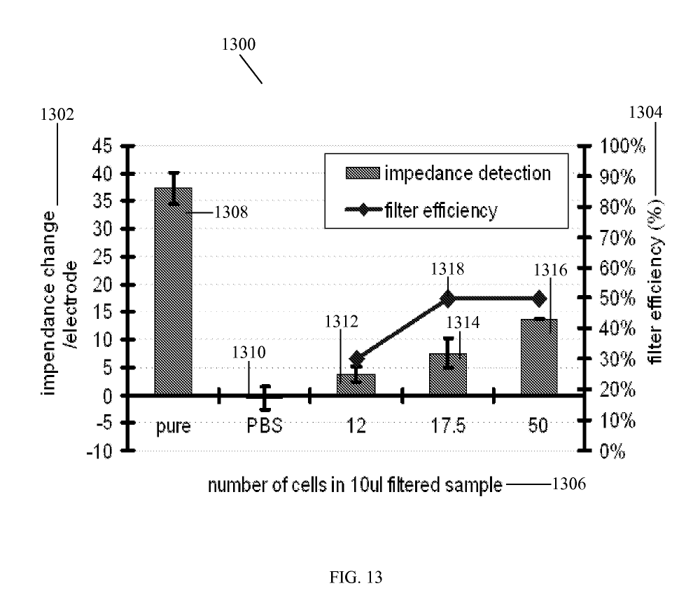

FIG. 13 shows a plot 1300 of impedance measurement and filter efficiency for a number of approximately 10 μl filtered samples provided to the microchip for detection, according to various embodiments. The plot 1300 is shown in terms of the impedance change per electrode 1302 and the filter efficiency 1304 against the number of cells in 10 μl filtered sample 1306.

FIG. 13 shows the impedance change per electrode 1302 obtained for a sample of pure cells 1308 without fluidics, a sample of PBS 1310 without cells, a sample containing about 12 cells 1312, a sample containing about 17 to 18 cells 1314 and a sample containing about 50 cells 1316.

The results show that the sample of pure cells 1308 shows a relatively high impedance change per electrode 1302 where the electrodes are saturated with cells while the sample of PBS 1310 shows an impedance change per electrode 1302 in the negative region. The impedance change per electrode 1302 observed for the sample containing about 12 cells 1312, the sample containing about 17 to 18 cells 1314 and the sample containing about 50 cells 1316, show a relatively highly sensitive system for detection of relatively small number of cells.

In order to increase the number of cells on the electrodes for the impedance measurement, a second batch or subsequent batches of samples may be added to the present batch.

FIG. 13 also shows that the filter efficiency 1304, as represented by the data points 1318, for the sample containing about 12 cells 1312, the sample containing about 17 to 18 cells 1314 and the sample containing about 50 cells 1316, is between about 30% to about 50%.

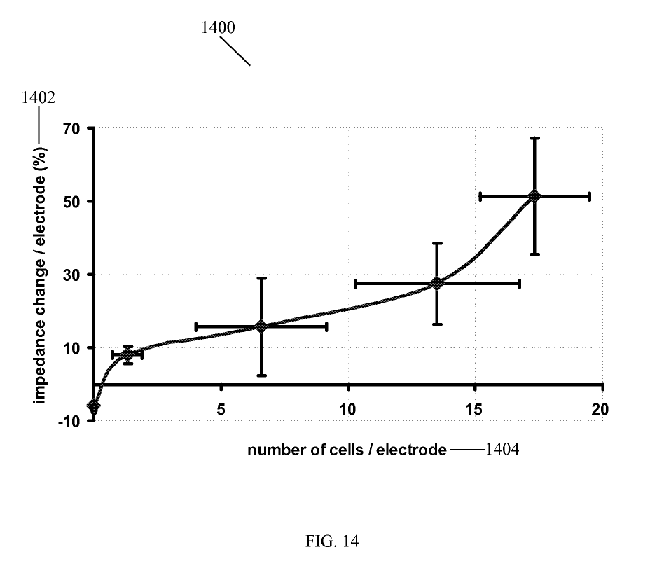

A measurement was also carried out to determine the detection sensitivity of the electrodes for different numbers of cells on the electrodes. A number of cells, ranging from 1-20, were positioned on the electrode and the impedance change per electrode was measured. The results are shown in FIG. 14 for a plot 1400 of impedance measurement. The plot 1400 is shown in terms of the percentage of the impedance change per electrode 1402 against the number of cells per electrode 1404. FIG. 14 shows that the microfluidic chip of various embodiments show a detection sensitivity of <10 cells.

An open-chamber concept detection chamber provides a relatively efficient transfer of cells to the detection chamber and a relatively efficient detection of cells in the detection chamber. The use of close-chamber flow-through fluidics at the detection region with the use of DEP to capture the cells passing through the microchannel on the microelectrode array or electrodes at the detection region may be considered but design constraints linked to the size of the electrodes and the multiplexing structures of the microchip may limit the use of DEP to a range where capture of the cells may not be possible. In order to determine this, a PDMS capping layer with no detection chamber or its associated pin was fabricated. In a trapping and release operation in accordance with various embodiments, cells flowing through the microchip were observed to flow at a relatively slow flow rate of <0.5 μl/min in the magnetic trapping region while employing the DEP process at the detection region at a voltage of about 10 V and a frequency of about 2 MHz. Such a relatively slow flow rate may not be practical for applications requiring processing of large volumes of samples. In addition, a relatively slower flow rate may be necessary for efficient trapping of the cells, which may not be compatible with the requirements for the release of the cells from the magnetic trapping region, while also increasing cell loss in the microchannels due to non-specific attachment of the cells to the walls of the microchannels. Accordingly, an open chamber, coupled with DEP, may be relatively more efficient to adequately capture and detect the cells in the detection region.

It may also be considered microfabricating magnets at the bottom of the detection chamber but the integration of the microfabricated magnets and the method of impedance detection may not be compatible, in addition to noting that the magnetic force induced by the magnets may be relatively weak to trap the cells on the electrodes.

It may also be considered providing an external magnet beneath the detection region but it may not be possible to localize the magnetic field so as to trap the cells specifically on the electrode as the competing forces between the DEP and the magnetic force may not be favorable to DEP. In order to determine this, a closed microchannel at the detection region was fabricated and it was observed that positioning the cells for detection may not be possible. Based on the above observation, magnetic trapping of the cells should be performed at another area on the microchip, thereby necessitating the transfer of cells from the magnetic trapping region to the detection region, in accordance with various embodiments.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A microfluidic system for detecting a biological entity in a sample volume, the microfluidic system comprising:
an inlet configured to receive the sample volume;
at least one microchannel in fluid communication with the inlet;
a magnetic trapping region comprising the at least one microchannel;
at least one detection region in fluid communication with the magnetic trapping region for detecting the biological entity to be detected; and
at least one outlet in fluid communication with the at least one detection region.

2. The microfluidic system according to claim 1, wherein the at least one microchannel comprises a shape selected from the group consisting of a serpentine shape, a meander shape, a spiral shape and any combination thereof.

3. The microfluidic system according to claim 2, wherein the at least one detection region comprises a microelectrode array.

4. The microfluidic system according to claim 3,
wherein the microelectrode array comprises a plurality of electrodes; and
wherein each of the plurality of electrodes has a dimension smaller than 4000 μm.

5. The microfluidic system according to claim 3, wherein the microelectrode array comprises sensor electrodes.

6. The microfluidic system according to claim 3, wherein a width of the at least one microchannel in the magnetic trapping region is relatively larger than a width of the at least one microchannel outside the magnetic trapping region.

7. The microfluidic system according to claim 6, further comprising:
a trapping chamber comprising the magnetic trapping region; and
a detection chamber comprising the at least one detection region.

8. The microfluidic system according to claim 7, wherein a volume of the detection chamber is approximately ten times larger than a volume of the trapping chamber.

9. The microfluidic system according to claim 1, wherein the magnetic trapping region is configured to reduce a flow rate in the at least one microchannel such that a magnetic force at the magnetic trapping region is configured to trap the biological entity.

10. The microfluidic system according to claim 1, further comprising:
at least one magnet;
wherein the at least one magnet is movably arranged, such that the at least one magnet in a first position is configured to trap the biological entity in the magnetic trapping region and in a second position is configured to release the biological entity.

11. The microfluidic system according to claim 1, wherein the biological entity is selected from the group consisting of a rare biomarker, a cell, a eukaryotic cell, a prokaryotic cell, a mammalian cell, a yeast cell, a tumour cell, a circulating tumour cell, a progenitor cell, an endothelial progenitor cell, a fetal cell, an organelle, a virus particle, a biopolymer, a polypeptide, a nucleic acid, a lipid, an oligosaccharide, and any combination thereof.

12. A microfluidic arrangement, comprising
a microfluidic system for detecting a biological entity in a sample volume, the microfluidic system comprising:
an inlet configured to receive the sample volume;
at least one microchannel in fluid communication with the inlet;
a magnetic trapping region comprising the at least one microchannel;
at least one detection region in fluid communication with the magnetic trapping region for detecting the biological entity to be detected; and
at least one outlet in fluid communication with the at least one detection region; and
a microchip being formed on or in the microfluidic system in a monolithically integrated manner.

13. The microfluidic arrangement according to claim 12, wherein the microchip comprises a capping layer.

14. An assembly for detecting a biological entity in a sample volume, the assembly comprising:
a microfluidic system for detecting a biological entity in a sample volume, the microfluidic system comprising:
an inlet configured to receive the sample volume;
at least one microchannel in fluid communication with the inlet;
a magnetic trapping region comprising the at least one microchannel;
at least one detection region in fluid communication with the magnetic trapping region for detecting the biological entity to be detected; and
at least one outlet in fluid communication with the at least one detection region; and
a magnetic labeling device configured to label the biological entity to be detected with magnetic beads;
wherein the magnetic labeling device is in fluid communication with the microfluidic system to supply the labeled biological entity to the microfluidic system.

15. A method for detecting a biological entity in a sample volume using a microfluidic system for detecting a biological entity in a sample volume, the microfluidic system comprising:
an inlet configured to receive the sample volume;
at least one microchannel in fluid communication with the inlet;
a magnetic trapping region comprising the at least one microchannel;
at least one detection region in fluid communication with the magnetic trapping region for detecting the biological entity to be detected; and
at least one outlet in fluid communication with the at least one detection region;
the method comprising:
providing the sample volume to the at least one microchannel via the inlet;
trapping the biological entity to be detected at the magnetic trapping region via a movably arranged magnet, wherein the magnetic trapping region is in fluid communication with the at least one microchannel;
removing the magnet from the magnetic trapping region;
transferring the biological entity from the magnetic trapping region to the at least one detection region; and
detecting the biological entity in the at least one detection region.

16. The method according to claim 15, wherein the method comprises a process selected from the group consisting of dielectrophoresis, capturing by antibodies, impedance measuring, and any combination thereof.

17. A method for manufacturing a microfluidic system, the method comprising:
providing a substrate;
thinning the substrate at a predetermined portion of the substrate from a first surface of the substrate;
forming a magnetic trapping region in a vicinity of the thinned portion of the substrate on a second surface of the substrate opposite the first surface;
forming at least one microchannel in fluid communication with the magnetic trapping region on the substrate;
forming at least one detection region in fluid communication with the magnetic trapping region;
forming at least one electrode in the at least one detection region; and
providing a capping layer on the substrate.

18. The method according to claim 17, wherein thinning the substrate is carried out by means of etching.

19. The method according to claim 17, wherein forming the at least one microchannel on the substrate is carried out using a photoresist, wherein the photoresist is spin-coated on the substrate and patterned using a lithography process.

20. The method according to claim 17, wherein forming the at least one electrode on the at least one detection region is carried out using metal deposition and patterning.
